(19) United States
(12) Patent Application Publication
(10) Pub. No.: US 2017/0030876 A1
(43) Pub. Date: Feb. 2, 2017

(54) Combinational Array Gas Sensor

(71) Applicant: Odosodo, Inc., Emeryville, CA (US)

(72) Inventors: Fanqing Chen, Moraga, CA (US); Xuhui Sun, Jiangsu (CN); David S. Alessio, Cupertino, CA (US)

(21) Appl. No.: 15/056,859

(22) Filed: Feb. 29, 2016

Related U.S. Application Data
(63) Continuation of application No. 13/843,699, filed on Mar. 15, 2013, now abandoned.

(51) Int. Cl. G01N 33/00; H01L 21/283; H01L 29/06; H01L 21/306; G01N 27/12; G01N 27/22

(52) U.S. Cl. CPC G01N 33/0031; G01N 27/12; G01N 27/227; H01L 29/0657; H01L 21/30604; H01L 21/283

(57) ABSTRACT

Described is a combinational array gas sensor. In one aspect is described as an apparatus for measuring a concentration of at least one gas in air comprising an integrated semiconductor sensor unit, the semiconductor sensor unit comprising a common substrate; a plurality of semiconductor sensors disposed over the common substrate, wherein each of the plurality of semiconductor sensors senses at least one of a plurality of different gases, wherein at least one of the plurality of sensors senses the at least one gas, and wherein each of the plurality of the semiconductor sensors include two electrodes and a plurality of semiconductor ridges disposed between the two electrodes, each of the plurality of semiconductor ridges being made of a same composition of semiconductor material, thereby allowing the air with the gas disposed therein to be proximate to each of the plurality of semiconductor ridges unless inhibited by an inhibitor material; and a circuit that uses a source current to pass a measurement current through at least some of the plurality of semiconductor sensors and cause outputting of at least one measurement signal from the plurality of semiconductor sensors.

### RELATED APPLICATIONS

This application is a continuation of U.S. patent application Ser. No. 13/843,699 filed on Mar. 15, 2013, which is incorporated in its entirety.

### BACKGROUND OF THE RELATED ART

Gas sensors are well known. Over the past few decades, with the growing need for high performance gas sensors, researchers and engineers have dedicated their effort to develop both materials and sensors with the characteristics of high sensitivity, good selectivity, and reliability.

Conventionally, many sensors are based on basic metal oxides thin films and nanomaterials due to their high surface area/volume ratio, such as hierarchical structure Nanomaterials (3D), Graphene, Nanosheet (2D), Nanowires, Nanobelts, Nanoribbons, MCNT/SCNT (1D), Nanoparticles (OD) or doped nanomaterials. Convention has also been to focus on getting high selectivity for a particular gas for gas detection and with ruling out other gas interference.

### SUMMARY

Described is a combinational array gas sensor in one aspect is described as an apparatus for measuring a concentration of at least one gas in air comprising an integrated semiconductor sensor unit, the semiconductor sensor unit comprising a common substrate; a plurality of semiconductor sensors disposed over the common substrate, wherein each of the plurality of semiconductor sensors senses at least one of a plurality of different gases, wherein at least one of the plurality of sensors senses the at least one gas, and wherein each of the plurality of the semiconductor sensors include two electrodes and a plurality of semiconductor ridges disposed between the two electrodes, each of the plurality of semiconductor ridges being made of a same composition of semiconductor material, thereby allowing the air with the gas disposed therein to be proximate to each of the plurality of semiconductor ridges unless inhibited by an inhibitor material; and a circuit that uses a source current to pass a measurement current through at least some of the plurality of semiconductor sensors and cause outputting of at least one measurement signal from the plurality of semiconductor sensors.

In another aspect is described as a method of making a semiconductor gas sensor comprising the steps of providing a substrate opening a cavity in the substrate; filling opposite sidewalls of the cavity and an adjacent top region with a conductor to form a pair of electrodes; and forming a plurality of semiconductor ridges disposed between the two electrodes within the cavity, each of the plurality of semiconductor ridges being made of a same composition of semiconductor material, thereby allowing the air with the gas disposed therein to be proximate to each of the plurality of semiconductor ridges.

A method of forming a semiconductor ridge having a predetermined composition and a predetermined length, width and depth for use as a gas sensor comprising the steps of, comprising the steps of forming a first layer of semiconductor material of a predetermined material to a predetermined thickness on a substrate; forming a second layer of semiconductor material of another predetermined material that is different than the first predetermined material to another predetermined thickness over the first layer of semiconductor material to form a composite layer; etching the composite layer to form the semiconductor ridge having the predetermined length, width, and exceeding the depth desired for the semiconductor ridge; and removing the semiconductor ridge from the substrate so that the semiconductor ridge results in the predetermined depth.

Further another aspect described is a method of measuring a concentration of at least one gas in air comprising introducing air into a semiconductor sensor unit; disposing the air proximate to a plurality of sensors within the semiconductor sensor unit, each of the sensors including a plurality of semiconductor ridges, the plurality of semiconductor ridges for each sensor being formed over a common substrate, parallel to each other and having opposite ends, with each connected between a pair of electrodes at the opposite ends thereof, each of the plurality of semiconductor ridges being made of a same composition of semiconductor material; obtaining a plurality of measurement signals from the plurality of semiconductor sensors using a circuit that passes a measurement current through the plurality of semiconductor sensors and cause outputting of the plurality of measurement signals; and analyzing the measurement signals using a detection algorithm to determine a concentration of the gas.

### BRIEF DESCRIPTION OF THE DRAWINGS

These and other aspects and features will become apparent to those of ordinary skill in the art upon review of the following description of specific embodiments of the invention in conjunction with the accompanying figures, wherein:

FIG. 1 is a top view of an example system configuration of two-layer and 8×8 array Combinational Array Sensor Device in accordance with this present invention, based on two layers.

FIG. 2 shows a top view of an example system configuration of two-layer and 8×8 array Combinational Array Sensor Device in accordance with the present invention. FIG. 3(A-B) Cross-section of an example system configuration of two-layer and 8×8 array Combinational Array Sensor Device

FIG. 4 shows a cross-section of an example system configuration of multi-layer and 8×8 array Combinational Array Sensor Device.

FIG. 5A: Top view of an example system configuration of an individual site on the chip array.

FIG. 5B: Cross-section parallel to Y direction of an example system configuration of an individual site on the chip array.

FIG. 5C: Cross-section parallel to X direction of an example system configuration of an individual site on the chip array.

FIG. 6 Show structure of each vertical nanobelt in cavity.

FIG. 7 Shows elemental metals that are used for metal oxide sensing materials in the periodic table, with those greyed out not typically used for metal oxide sensing materials.

FIG. 8 A chart of an example value system of x/y of CrxOy expressed by horizontal axis.

FIGS. 9A-9I Show top views schematic diagram illustrating the fabrication process of the first layer of combinational array sensor device.

FIGS. 10A-10H Show top views schematic diagram illustrating the fabrication process of the second layer of combinational array sensor device.

FIGS. 11A-11I Show side views schematic diagram illustrating the fabrication process of the first layer of combinational array sensor device.

FIGS. 12A-H Show side views schematic diagram illustrating the fabrication process of a portion of the second layer of combinational array sensor device.

FIG. 13 Show 16 kinds of Masks for the combinational array

FIGS. 14A-14M show cross-section views in the Y direction of semiconductor processing steps for forming the one-layer individual site on chip.

FIG. 15N Shows cross-section view in the X direction of the one-layer individual site on chip in the same view as FIG. 14M.

FIG. 16 Show top view of an example of the multi-layer of individual site on chip, X and Y are two directions that are perpendicular to one another in the horizontal plane. The gray part is the silicon substrate; the golden part is the Au or other metal thin film used as electrodes; the blue part is the silicon oxide used for the insulating barrier. L is the length of the cavity.

FIG. 17A Show an example of cross-section view in the Y direction (in FIG. 16) of the Multi-layer of individual site on chip. L is the length of the cavity.

FIG. 17B Show an example of cross-section view in the X direction (in FIG. 16) of the Multi-layer of individual site on chip. W is the width of each sensing material valley.

FIG. 18A Show an example of cross-section view in the Y direction of the Multi-layer of individual site on chip after it is annealed.

FIG. 18B Show an example of cross-section view in the Y direction of the Multi-layer of individual site on chip after it is annealed.

FIG. 19A shows a cross-view of a cavity with 45° angle via etching the silicon with (100) crystal direction using patterned Photorisist.

FIG. 19B shows a top view of a cavity with 45° angle via etching the silicon with (100) crystal direction using patterned Photorisist.

FIG. 20A-20B Scanning Electron Micrograph (SEM) images of cavity with 45° angle with gold electrode.

FIG. 21 shows a top view of cavity with X length and Y width.

FIG. 22 shows the relationship of the silicon cavity width and length vs etching time.

FIG. 23A-23E Show different silicon cavities depth pictures etched by 33% KOH etching solution at 50° with different etching time.

FIG. 24 shows an example of relationship schematic between silicon cavity depth and etching time.

FIG. 25 Photon microscope picture of cavity with ear-type Au-electrode:

FIG. 26A-26D Show different width electrode ear pictures.

FIG. 27A-27B Show different shapes of electrode bonding side, square and rectangle

FIG. 28 is a mask for a cavity of an individual site on chip.

FIG. 29 Shows an example of Masks for electrode of individual site on chip.

FIG. 30 Shows an example of Masks for sensing materials.

FIG. 31 Shows an example of Masks for Ridge Pattern

FIG. 32A-32B Cross-section of two layer individual unit of combinational array sensor.

FIG. 33 Top view of two layer individual unit of combinational array sensor.

FIG. 34 Show a 3D view of one valley of sensing material in cavity of individual unit on chip.

FIGS. 35-36 Show examples of whole sensing material in cavity.

FIG. 37 Show the 3D view of the diffusion of gas between vertical sensing materials.

FIG. 38 Show the side view of the diffusion of gas between vertical sensing materials.

FIG. 39 Show the side view of the diffusion of gas between vertical sensing materials.

FIGS. 40A-B illustrate a sensor matrix and output circuit relating thereto.

### DETAILED DESCRIPTION OF THE PREFERRED EMBODIMENTS

The architecture of sensing elements is recognized herein as a very important factor influencing the performance of gas sensors. The combinational array sensor embodiments described herein focus on that, as well as can be made using conventional semiconductor fabrication technologies.

The combinational array sensor described herein with many kinds of different sensing units can detect different aspects of a gas (i.e. smell), allowing for identification at the molecular level. Responses to a particular gas of each kind of sensor unit are different. Different responses to gas mixture are obtained by the different sensor units of the combinational array sensor, and by integrating these different responses of all sensor units a better sensing is achieved.

Fundamental architectural aspects of the described combinational array sensor are shown by FIG. 1, which is a top view of an example system configuration of two-layer and 8×8 array Combinational Array Sensor Device in accordance with this present invention, based on two layers.

In FIG. 1. Each mall square ( [symbol] ) stands for a sensing unit on chip array; each right-angled triangle ( [symbol] ) stands for one kind of sensing material. Different colors (shown here and throughout as different shades of grey, with different shades of grey being apparent in different rows and columns, thus allowing for different shades within each triangle, though also referred to hereinafter as different colors) stand for different kinds of materials. Each Combinational Array Sensor Device may have many sensing unit with different kinds of materials. Each individual sensor unit on the chip array has different sensing material from others. In the column direction, there are 8 columns from 1 to 8; In the row direction, there are 8 rows from A to H.

A prior art single sensor may have “perfect” sensitivity for one analyte but poor selectivity; it may also show sensitivity to other gasses. But in the combinational array sensors described herein containing many sensing units, while each sensor unit is dedicated to sending a specific gas, the many different kinds of sensor units allow for sensing across a range, which allows for both high sensitivity and high selectivity.

Significant aspects with respect to this section are:
There are many parameters which can be changed to get many variations of Combinational Array sensors (Number of layers, the width of thin film nanosheet, the thickness of sensing materials, kinds of materials, compound modes of the same materials, the size of combinational array and so on)
This compound mode of combination array sensor can detect different kind of gases simultaneously.
a complicated Combinational Array can be fabricated using conventional semiconductor fabrication methodologies, as described further herein.

### 2.1 Combination Array

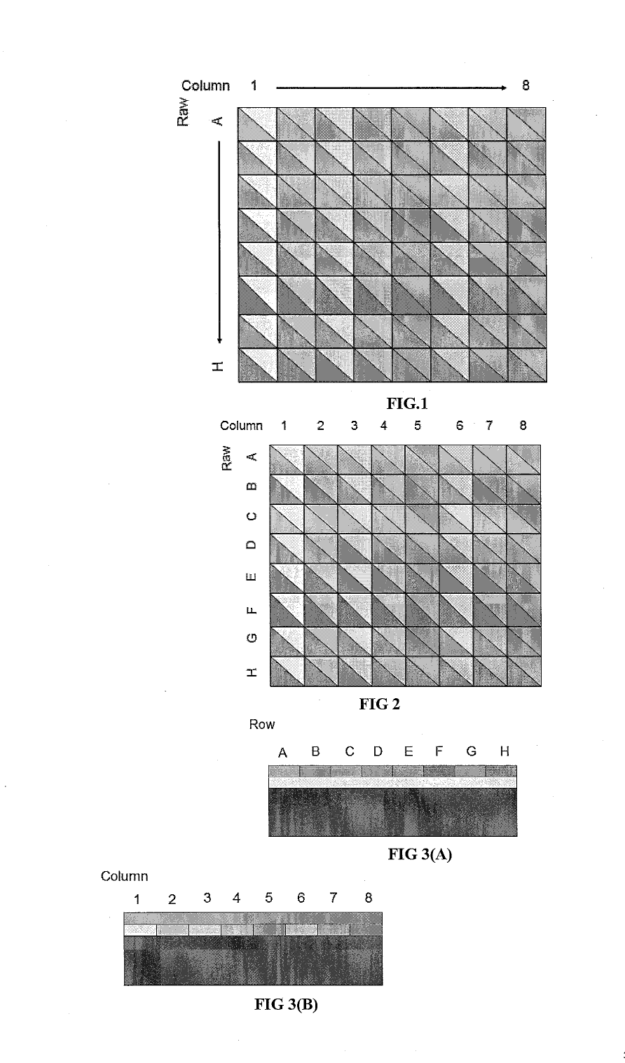

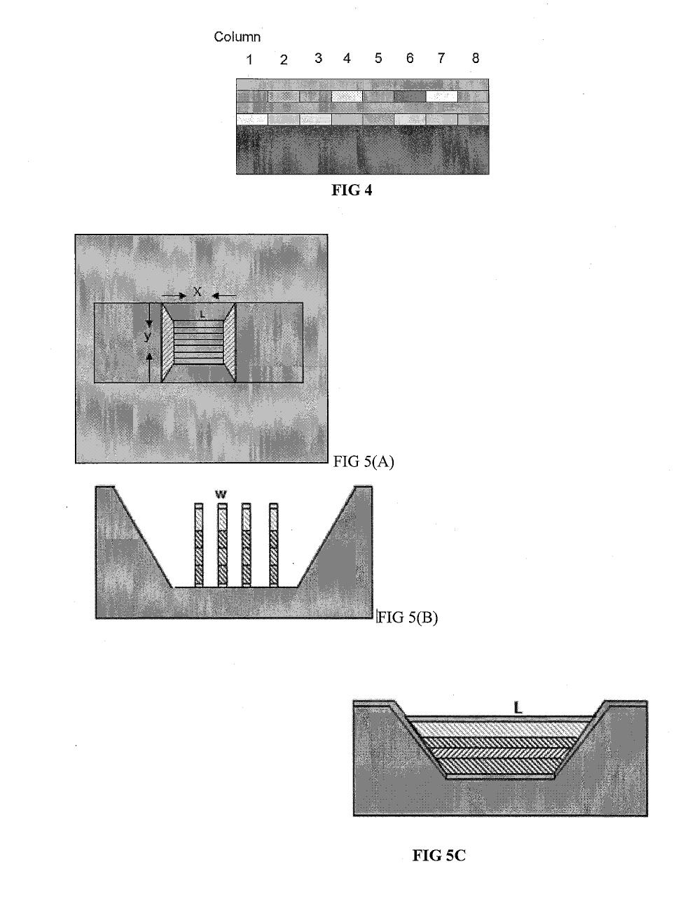

Example 1: Two-Layers sensing materials combination arrays.
Example 2: Multi-layer sensing materials combination arrays (Four layers).
FIG. 2 is a top view of an example system configuration of two-layer and 8×8 array Combinational Array Sensor Device:
Each mall square ( [symbol] ) stands for a sensing unit on chip array; each right-angled triangle ( [symbol] ) stands for one kind of sensing material.
Different colors stand for different sensor unit. Each sensor unit has a kind of mixed material and each sensor unit has different mixed material from others. Each Row and Column has 8 kinds of different mixed materials. So if there is 8×8 (N) array, we can get 64 kinds of mixed materials.
The materials in the same row or column of the same layer are the same materials.
   FIG. 3 (A-B) FIG. 3(A-B) Cross-section of an example system configuration of two-layer and 8×8 array Combinational Array Sensor Device
The gray color layer stands for the substrate, and the upper two layers stand for sensing materials.
The numbers of row and column are not limited to 8, this assembly can also be multiplied into a 10×10 array (or bigger array, it can range from 2 to 100) in Combination Array Sensor.
The number of row is not only the same as column, but can be different from column.
The number of layer are not limited to 2, other number can be used in Combination Array (the range can from 1 to 100 or bigger).
The size of Combinational Array is not limited. It can be from several millimeters to several centimeters even bigger.
   The response of each sensor unit to each kind of gas can be more or less expressed, and these responses differ in many ways.
   FIG. 4 shows a cross-section of an example system configuration of multi-layer and 8×8 array Combinational Array Sensor Device. As shown, an array of this type can be used in placing multiple sensing units in the same chip, and multiple electronic measurement units can be connected to electrodes for arrayed measurement, as described herein.

### 2.2 Individual Site on the Chip Array

### 2.2.1 Structure of Each Individual Site on the Chip Array

Example: Multi-Layer sensing materials (Four layers).
FIG. 5A is a top view of an example system configuration of an individual site on the chip array.
The gray part stands for the substrate, the upper golden layer stands for gold or other metal electrodes, and the blue layer in the bottom of cavity is the silicon oxide valley array insulation layer on the top of sensing materials.
x and y stand for the length and the width of the cavity.
L stands for the length of the nanosheet sensing materials in the cavity.
   FIG. 5B is a cross-section parallel to Y direction of an example system configuration of an individual site on the chip array.
The gray layer stands for the substrate, and the blue layers in the bottom and top of the sensing materials layers are silicon oxide insulation layers. Between these two insulation layers are sensing materials, different colors stand for different sensing materials. There is a vertical valley array of sensing materials in the cavity, and each vertical valley array has many vertical nanosheets.
W stands for the width of each vertical nanobelt sensing materials in the cavity.
   FIG. 5C is a cross-section parallel with X direction of an example system configuration of an individual site on the chip array.
The gray layer stands for the substrate, the upper golden layer stands for gold or other metal electrodes, and the blue layers in the bottom and top of sensing materials are silicon oxide insulation layers. Between these two insulation layers are sensing materials, different colors stand for different sensing materials.
W stands for the width of the nanosheet sensing materials in the cavity.
   Significant aspects with respect to this section are:
Each individual site on chip array has a Cavity:
The size of cavity is not limited, and it can be changed (it can range from several micrometers to several millimeters). The smaller sizes of cavities are, the higher density of individual sites is, and the better sensitivity data we will get.
The shape of cavity is not limited to square, other basic shapes of cavity can also be used in the individual site, and it is determined by the mask of cavity.
The depth of cavity is not limited, and it can be changed, and it is determined by the time of etched.
The cavity is consisted of slant surfaces. The angles of cavity surfaces are not limited, it can be changed, and it is determined by the crystal face orientation direction of substrate.
The shape of sensing materials in cavity is preferably thin film nanosheet.
The width of thin film vertical valley nanobelts array is not limited and can be changed (it can change from 5 nm to 1000 nm)
The thickness of thin film vertical valley nanobelts is not limited and can be changed (it can change from 5 nm to 1000 nm)
The kind of material of thin film vertical valley nanobelts can be changed.
The thickness of silicon oxide is about 5 nm, but is not limited, and it can be changed.
Each individual site on chip array has an ear-type electrode (Example of the cavity with Au-electrode ear-type)
Each individual site on chip array has one kind of composite material which is comprised of many kinds of materials.
The size of Combinational Array is not limited. It can from several millimeters to several centimeters.

### 2.2.2 Structure of Each Vertical Nanobelt Sensing Materials in Cavity of Individual Site on the Chip Array

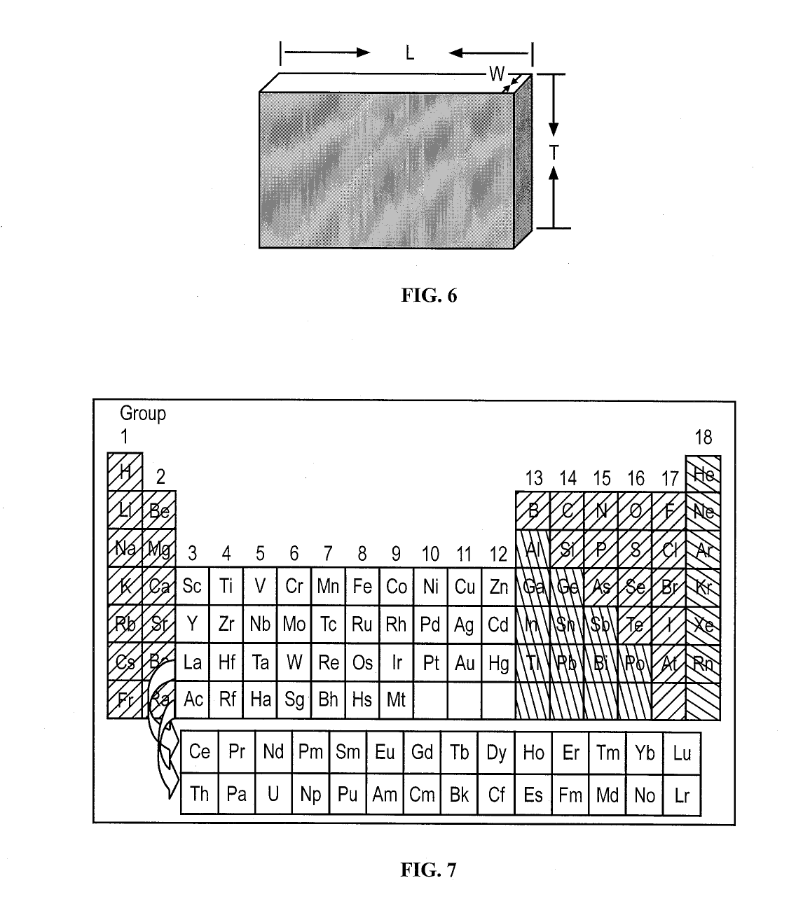

The vertical nanobelt sensing materials of the array in a cavity is shown by FIG. 6. The structure of each vertical nanobelt in cavity is express as:
L is the length of the vertical nanobelt; W is the width of the vertical nanobelt; T is the thickness of the vertical nanobelt.
L is the same value as the length of cavity, and it can be changed by changing the cavity size.
W can be changed by using different Ridge and valley Masks
T (Thickness of sensing materials)
   a). General T/W is 2/1
   b). The range of T/W is from 1:1 to 100:1
   c). The range of thickness is from 5 nm to 1000 nm.
      Parameter can be changed to get different forms of this vertical valley nanobelts array.

### 3. Materials Used for the Combination Array

Detecting gases is very important because it is necessary in many different fields. Over the past few decades, with the growing need for high performance gas sensors, more and more materials have been synthesized used for sensing materials. Because of the mechanisms for recognizing the gases to be determined include absorption processes and specific recognition for the formation of supramolecules or covalent bonds between the sensor and the analyte, many studies have also focused on reducing the size of the sensing materials in the form of nanoparticles or nanowires². Till now, most sensors were based on basic metal oxides thin films and nanomaterials³ due to their high surface area/volume ratio. Accordingly, many combinations of materials can be used for the combinational array sensors described herein. The following three type materials are materials that have been recognized as being most significant for use in the combinational array sensors described herein.

### 3.1 Basic Metal Chalcogenide Film.

Metal Chalcogenides possess a broad range of electronic, chemical, and physical properties that are often highly sensitive to changes in their chemical environment. The metal chalcogenides can be expressed by the following:

ⁱMeₓ^{j}Ch_{y}

where Me is the metal; i is the atomic number of the metal; Ch is the Chalcogen; j is the atomic number of the Chalcogen; x and y are the number of the metal and Chalcogen atoms respectively in each Metal Chalcogenide unit cell.

In these Metal Chalcogenide materials used for sensing, Metal Oxides are used in certain embodiments; other embodiments use Metal Chalcogenides, such as CdTe, CdSe, CdS, as sensing materials.

### 3.1.1 Metal Oxide Films

For this following expression:

ⁱMeₓ^{j}Ch_{y}

When j is 16, metal oxides are obtained, which can be expressed by the following:

ⁱMeₓO_{y} (“Metal Oxides”)
“i” is variable, different ‘i’ stands for different material in ⁱMeₓO_{y}, and allows for obtaining many kinds of basic metal oxides.
Example: When i=50, ⁱMeₓO_{y} is SnₓO_{y}
   When i=51, ⁱMeₓO_{y} is SbₓO_{y}
   . . .
      The metal is not limited to element of one Group (Group 11 or Group 12) of periodic table, other Groups of periodic table are also can be used for the suitable materials for sensing materials, and alloys or mixtures thereof. They can be the following:
      i. Group3-7 and Group11-12:
MnO₂, ZnO, WO₃, Sc₂O₃, TiO₂, V₂O₅, MnO₂, MoO₂ . . .
   ii. Group8-10
Co₂O₃, Ni₂O₃, Fe₂O₃, RuO₂, Rh₂O_{3y} PdO₂ . . .
   iii. Group13-16 Post Transition Metal Element
SnO₂, In₂O₃, Ga₂O₃, GeO₂, Sb₂O₃ . . .

TABLE 1
The form of an example system of Groups of periodic
table of basic Metal Oxide film which can be
used for Combinational Array Sensor

| Row | R(1) | R(2) | R(3) | R(4) | R(5) | R(6) | R(7) | R(8) | . . . |
|---|---|---|---|---|---|---|---|---|---|
| i | 21 | 22 | 23 | 24 | 25 | 26 | 27 | 28 | . . . |
| Transition | Sc | Ti | V | Cr | Mn | Fe | Co | Ni | . . . |
| Metal | | | | | | | | | |
| Element | | | | | | | | | |
| i | 13 | 31 | 32 | 49 | 50 | 51 | 81 | 82 | . . . |
| Post | Al | Ga | Ge | In | Sn | Sb | Tl | Pb | . . . |
| Transition | | | | | | | | | |
| Element | | | | | | | | | |

FIG. 7 Shows metals that are usually used for metal oxide sensing materials in the periodic table, with those greyed out not typically used for metal oxide sensing materials.
“x” and “y” are either the same or different ones.
Example: When i=50, (MeₓO_{y}═SnₓO_{y})
   x=1, y=1 (SnO)
or x=1, y=2 (SnO₂)
“x” and “y” are both variables.
x/y is not limited to theoretical value, other value can be used for Combinational Array Sensor in this patent we present.
Example 1: When i=50, (MeₓO_{y}═SnₓO_{y})
   The range of x/y can be the following:

TABLE 2
The form of an example value system of x/y of SnxOy.

| x/y | 0 | 0~1/2 | 1/2 | 1/2~1/1 | 1/1 | >1/1 |
|---|---|---|---|---|---|---|
| Material | O₂ | O₂~SnO₂ | SnO₂ | SnO₂~SnO | SnO | SnO~Sn |

Example 2: When i=23, (MeₓO_{y}═CrₓO_{y}) the range of x/y can be the following:

TABLE 3
The form of an example value system of x/y of CrₓO_{y}.

| x/y | 0-1/3 | 1/3 | 1/3-2/5 | 2/5 | 2/5-1/2 | 1/2 | 1/2-2/3 | 2/3 | 2/3-1/1 | 1/1 | 1/1-2/1 | 2/1 | >2/1 |
|---|---|---|---|---|---|---|---|---|---|---|---|---|---|
| Mat'l | O₂ to | CrO₃ | CrO₃ to | Cr₂O₅ | Cr₂O₅ to | CrO₂ | CrO₂ to | Cr₂O₃ | Cr₂O₃ to | CrO | CrO to | Cr₂O | Cr₂O to |
| | CrO₃ | | Cr₂O₅ | | CrO₂ | | Cr₂O₃ | | CrO | | Cr₂O | | Metal |

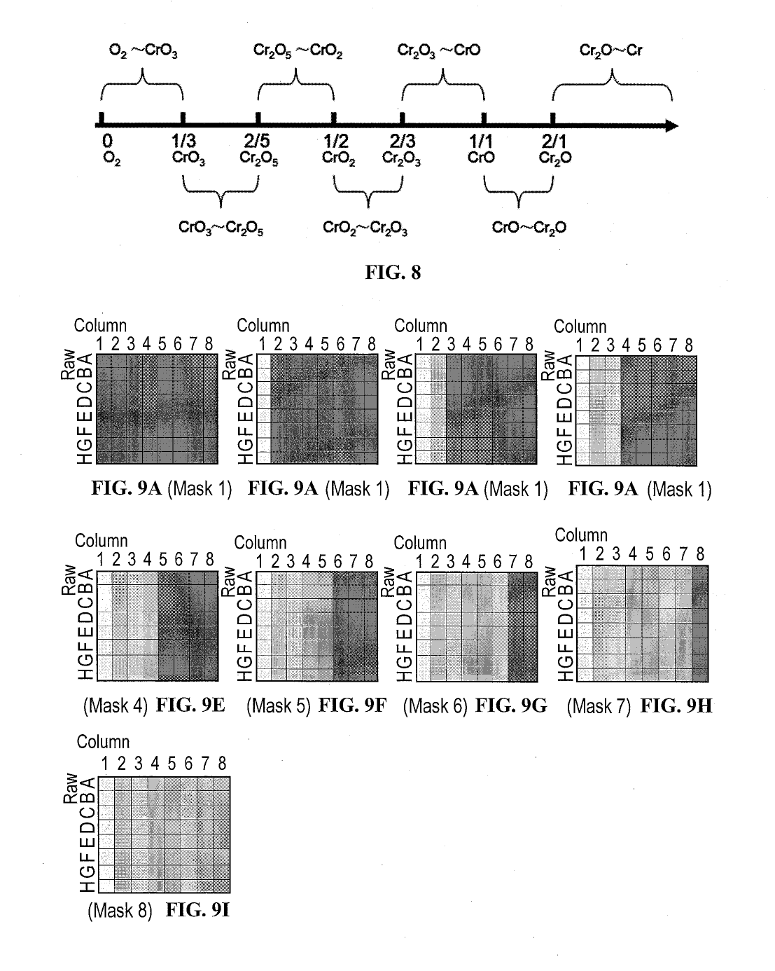

FIG. 8 A chart of an example value system of x/y of CrxOy expressed by horizontal axis.
There are two limit values in FIG. 8. When value of x/y is zero, the material is oxygen. When value of x/y is infinite, the material is chromium (metal). The value of x/y is not limited to theoretical value; every value of x/y can be got by changing the content of each elements. As such various kinds of materials are obtained by changing the value of x/y although the value of “i” is fixed value.
Just as:
   Theoretical value of x/y: SnO₂, SnO, Cr₂O₃, GeO₂ . . .
Non-theoretical value of x/y: SnO₃, SnO₄, Cr₂O₇ . . .

### 3.1.2 Other Metal Chalcogenide Films

Other Metal Chalcogenide materials such as CdTe, CdSe, CdS, also have very high sensing performance. CdTe alloyed doped with Cl, Hg, or Zn forms an excellent radiation detector, HgCdTe is sensitive to the widest range of IR.

### 3.2 Nanomaterials:

Nanostructure materials are a type of material that is particularly applicable with respect to the combination array described herein. They have high surface area/volume ratio, and as such a significant fraction of the atoms (or molecules) are surface atoms that can participate in surface reactions. This favors the adsorption of gases on the sensor and can increase the sensitivity of the device because the interaction between the analytes and the sensing part is higher. Nanostructure materials (nanomaterials) can also be used to reduce working temperatures and they consume less power and are safer to operate.

They can include the following:
Hierarchical structure Nanomaterials⁵, Porous material (3D)⁶ . . .
   Graphene, Nanosheet (2D)⁷ . . . .
   Nanowires^{2,8,9}, Nanobelts, Nanoribbons, MCNT/SCNT (1D)^{10,11} . . .
Nanoparticles (0D) . . .
   . . .

The structures of 3D nanomaterials used for sensing materials of Combinational Array sensor are also other structures (3D regular holes shape material and so on) involved in this patent. The structures of 2D nanomaterials are also nano thin film materials involved in this patent. All materials which can be used for sensing are also involved in our patent. For the structure of OD nanomaterials also can be Nanoflower, octagonal structure and so on.

Significant aspects with respect to this section are:
Nanomaterials are combined with each other and also combined with other kinds of sensing materials to obtain composite materials in each sensing unit, that have higher performance.

### 3.4 Doped Materials

In many gas sensors, the conductivity response is determined by the efficiency of catalytic reactions with detected gas participation, taking place at the surface of gas-sensing material. Therefore, control of catalytic activity of gas sensor material is one of the most commonly used means to enhance the performances of gas sensors. Doping is an important technique utilized to improve gas sensing properties, where the dopant atoms are believed to act as activators for surface reactions. So nowadays more and more metal-doping materials have been synthesized used for sensing. They have high performances for detecting some kinds of harmful gas, because noble metals are high-effective oxidation catalysts and this ability can be used to enhance the reactions on gas sensor surfaces.

They can be the following:
Metal-Doped ⁱMeₓ^{j}Ch_{y} (NW/NS/NF), SWCNT/MWCNT,

### Graphene . . .

a). Noble Metal-Doping (Pt, Pd, Au, Ag, Rh, Os, Ir, Ru)
b). Other metal-Doping (Fe, Co, Ni, Sb . . . . )
c). Lanthanide-Doping (La, Ce, Pr, Nd, Eu . . . . )
   Metal-Doped ⁱMeₓⁱCh_{y} (NW/NS/NF), SWCNT/MWCNT, Graphene
   Polymer-Doped
      . . .
   Doping metals and metal oxides and other materials in sensing materials is considered as an effective and simple way to improve the gas-sensing properties of pure sensing materials by increasing the sensitivity, reducing the operating temperature, decreasing the response and recovering time, as well as increasing the selectivity

### 4. Fabrication of Combinational Array

The method can allow high density array to be fabricated with relative small number of masks and lithography steps (Making N2 or N3 type of array with 2N and 3N masks). As illustrated in the cross sections of FIGS. 5A-5C, multiple layers can be used to create the sensor device, with 4 layers being shown in FIGS. 5A-5C.
Fabrication of the core of a two layer sensor is described with reference to the following figures and specific sized sensor, although it is understood that this can be adapted to other sensor sizes and dimensions, as noted above previously.

### Layer 1:

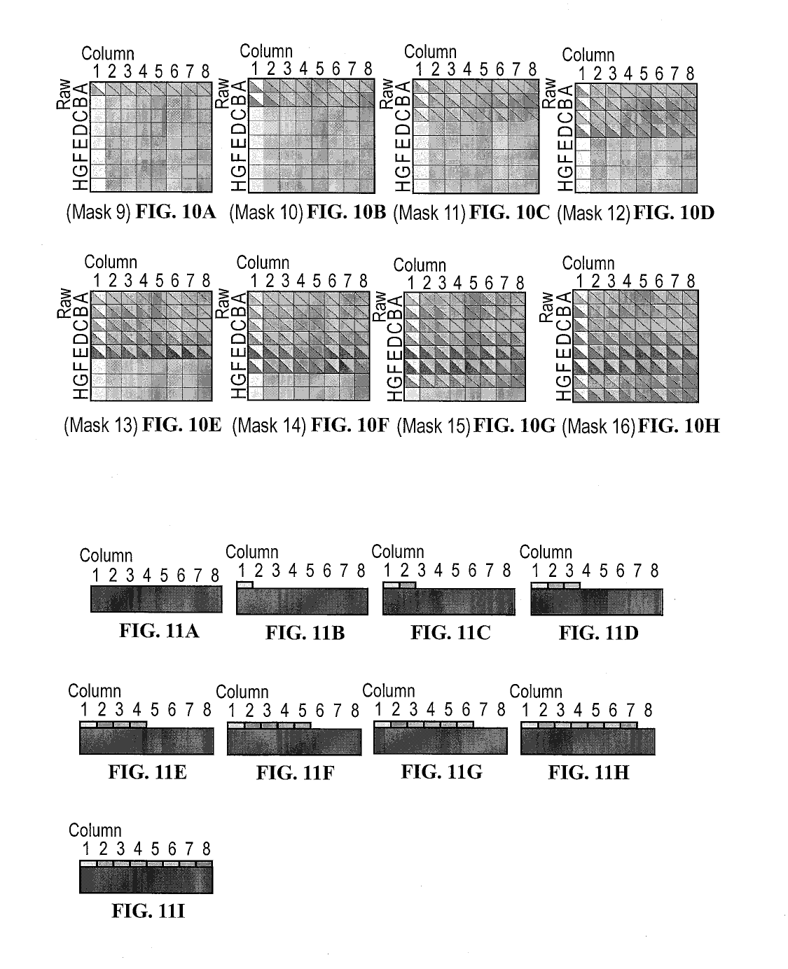

FIGS. 9A-9I Show top views schematic diagram illustrating the fabrication process of the first layer of combinational array sensor device.
FIGS. 10A-10H Show top views schematic diagram illustrating the fabrication process of the second layer of combinational array sensor device.
FIGS. 11A-11I Show side views schematic diagram illustrating the fabrication process of the first layer of combinational array sensor device.

### Layer 2:

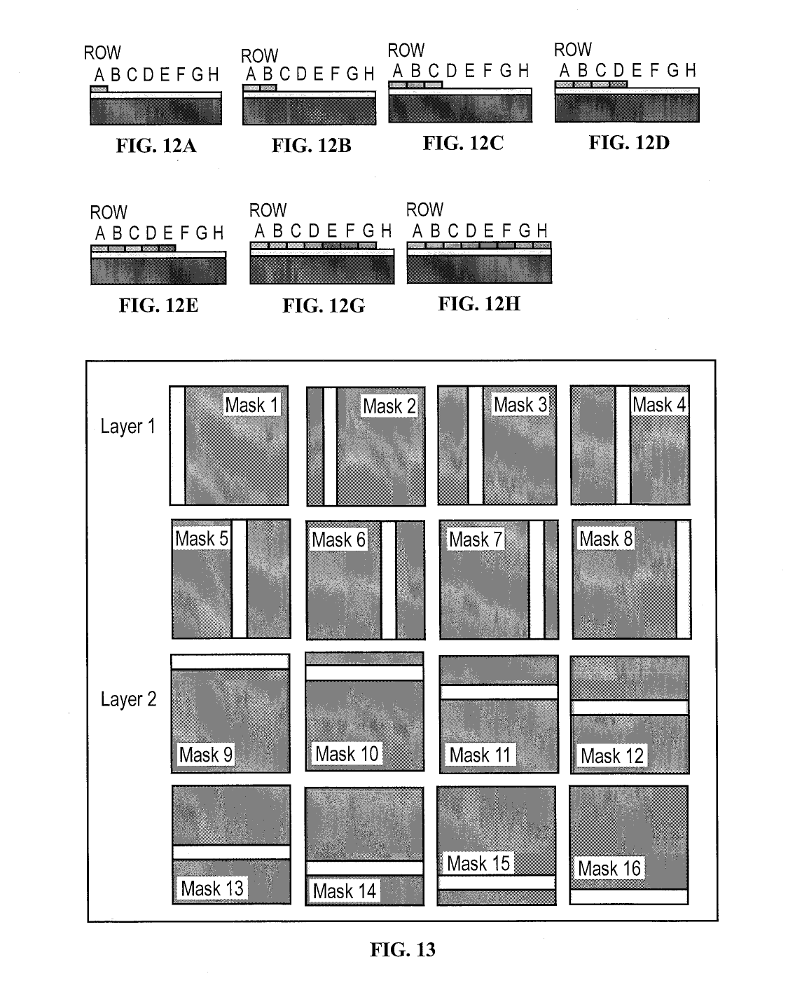

FIGS. 12A-D and 12-H Show side views schematic diagram illustrating the fabrication process of a portion of the second layer of combinational array sensor device.
Described is a 8×8 combinational array chip, which means that there are 8 rows (from A to H) and 8 columns (from 1 to 8) in the combinational array chip. So there are 64 individual units on this array chip. Each individual unit has a different material from others. So there will be 64 kinds of sensor units. Gray layer stands for the substrate, and the top two layers are two layers sensing materials. On the first sensing material layer, there are 8 kinds of materials deposited. They are deposited from column 1 to 8. For the second sensing material layer, there are also 8 kinds of materials deposited from raw 1 to 8.
Fabrication process:

### Layer 1:

### FIG. 11A-I:

A) Silicon wafer ( [symbol] ); B) Sensing material 1 ( [symbol] ) deposition on a Si wafer of column 1 using mask 1; C) Sensing material 2 ( [symbol] ) deposition on a Si wafer of column 2 using mask 2; D) Sensing material 3 ( [symbol] ) deposition on a Si wafer of column 3 using mask 3; E) Sensing material 4 ( [symbol] ) deposition on a Si wafer of column 4 using mask 4; F) Sensing material 5 ( [symbol] ) deposition on a Si wafer of column 5 using mask 5; G) Sensing material 6 ( [symbol] ) deposition on a Si wafer of column 6 using mask 6; H) Sensing material 7 ( [symbol] ) deposition on a Si wafer of column 7 using mask 7; I) Sensing material 8 ( [symbol] ) deposition on a Si wafer of column 8 using mask 8.

### Layer 2:

FIG. 12A-D (portion shown: (A)-(D) Sensing material 9-16 deposition on Si wafer from Raw A to Raw H using mask 9 to 16. Each steps of fabrication process is similar to fabrication process of the first layer.

Significant aspects with respect to this section are:
In the steps of forming the combinational array sensor device, the order of steps in row is not limited from Row A to Row H. Each layer can be finished in Row direction at random.
In the steps of forming the combinational array sensor device, the order of steps in column is not limited from Column 1 to Column 8. Each layer can be finished in column direction at random.
In the steps of forming the combinational array sensor device, the order of steps are not limited from Column to Row, the order of steps from Raw to Column can also be used to finish combinational array.
Making 2 layers (N2, N=8) type of array with 16 kinds of masks, we can get 64 kinds of different materials.
Making 2 layers (N2) type of array with 2N masks.
The numbers of Row and Column are not limited to 8. The range can be from 1 to 100 even 1000.
The numbers of Row and Column are not limited to the same as each other, they can be different form each other in our patent.
The numbers of layers is not limited. The range can be from 1 to 100.

### 5.1.2 Selection of Mask for Sensing Material of Combinational Array.

We take 8×8 (N=8) array on chip for example.

TABLE 4
The form of an example relationship system among Number of layers,
Times of using Masks and Compound modes of Materials.

| Number of | | Times of | Compound modes |
|---|---|---|---|
| layers | Number of Masks | using Masks | of Materials |
| 1 | 8 (N) | 8 (N) | 8 (N¹) |
| 2 | 16 (2N) | 16 (2N) | 64 (N²) |
| 3 | 16 (2N) | 24 (3N) | 512 (N³) |
| 4 | 16 (2N) | 32 (4N) | 4096 (N⁴) |
| . . . | | . . . | . . . |
| n | 16 (2N) | 8n (n * N) | 8ⁿ (Nⁿ) |

Masks for lhe combinational array:
   Example: 2-Layer 8×8 (N=8) array on chip, we need 16(2N) kinds of Masks, just as following:
      FIG. 13 Show 16 kinds of Masks for the combinational array. From FIG. 13, we can see that there are 16 kinds of masks for a 2-Layer 8×8 (N=8) combinational array chip. The gray part of the mask is lightproof part, and the white part is nonopaque part. If there are more layers than 2, the masks can be reused for several times, and there is no need to use more masks. The sensing materials are changed to get different combinational modes of mixed materials.
      Significant aspects with respect to this section are:

These masks above are just used for lithography of positive photoresist. For lithography of negative photoresist, masks are the opposite, that is, the gray part of the mask is nonopaque part, and the white part is lightproof part. The shape is the same.

### 5.2 Fabrication of Individual Site on Chip

5.2.1 Fabrication Process of the Individual Site on Chip.

### One Layer:

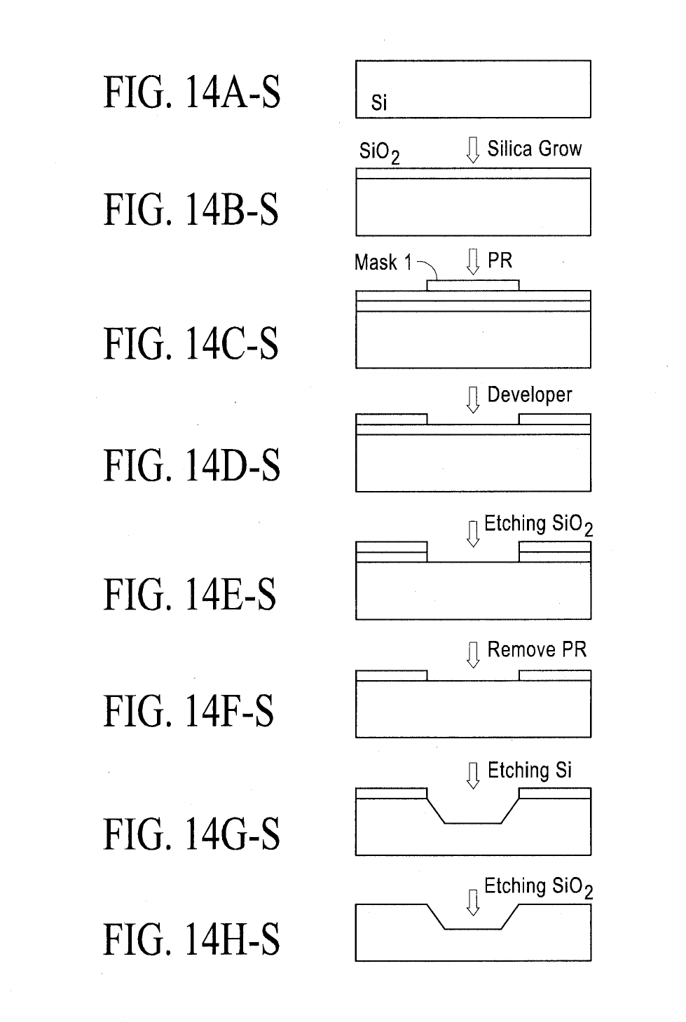

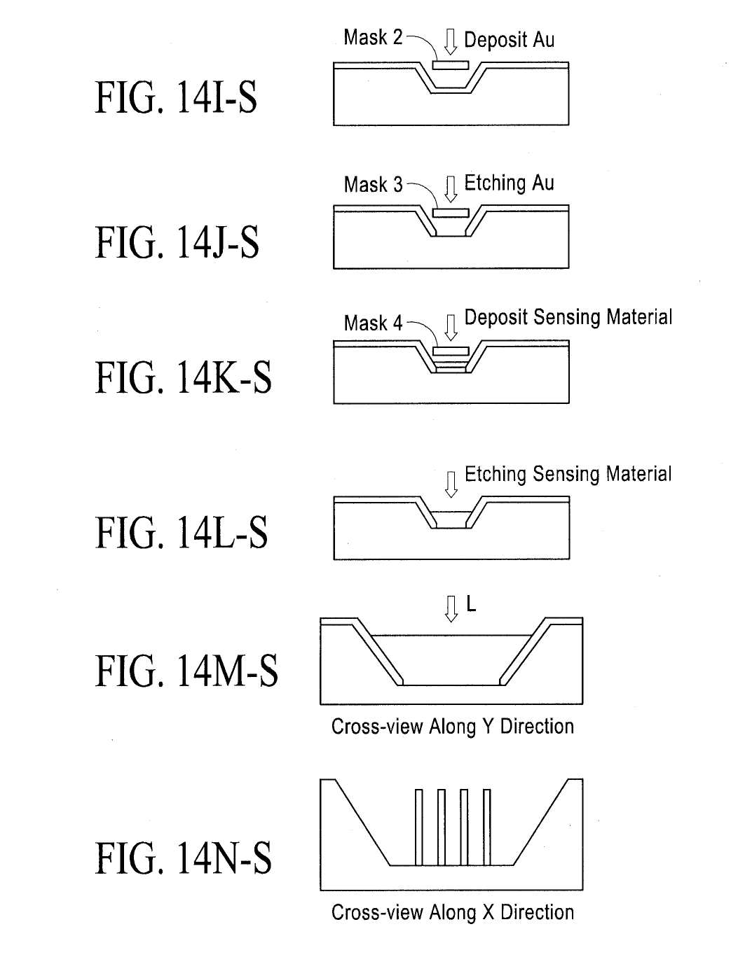

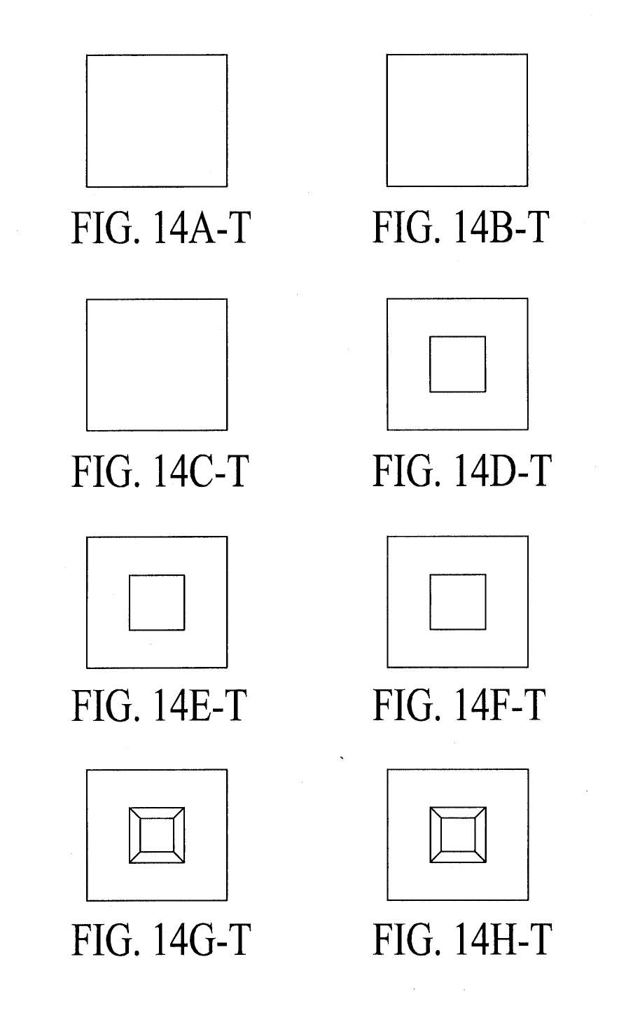

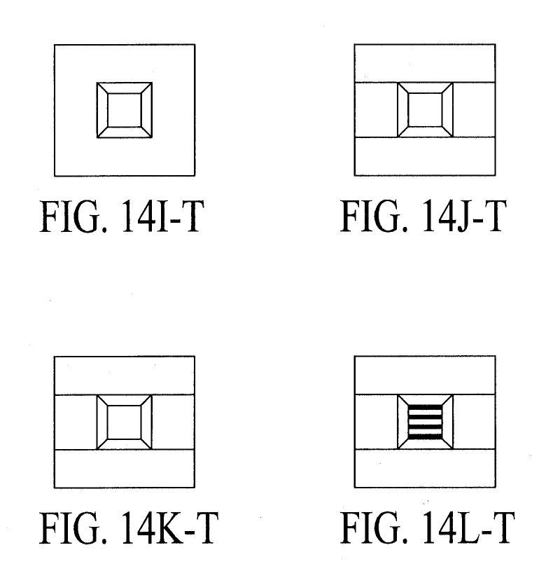

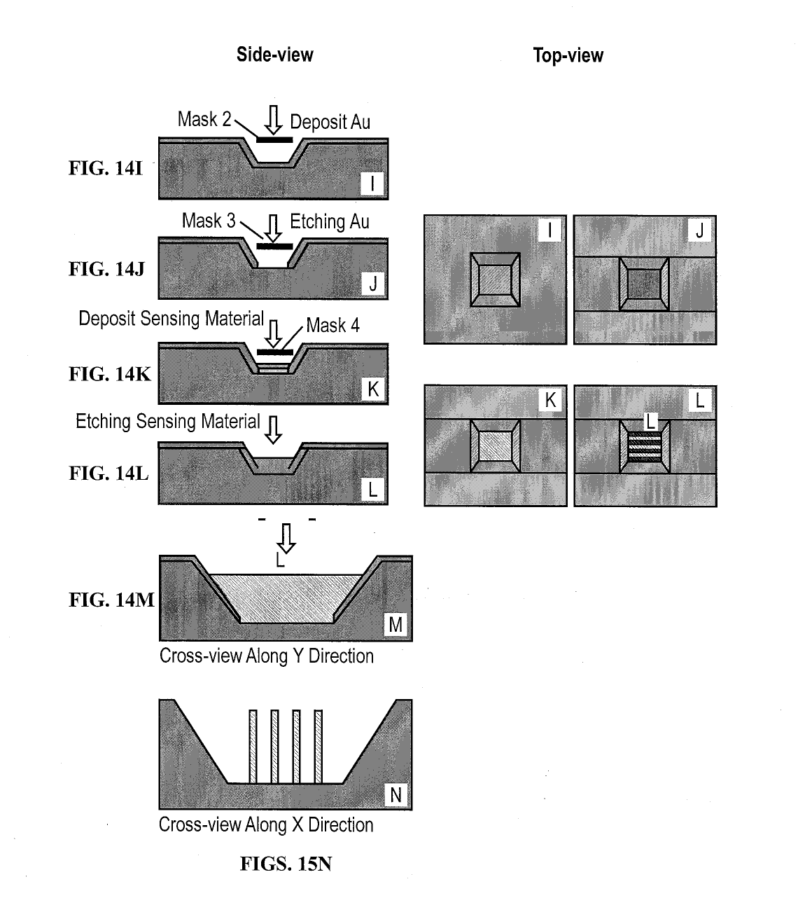

FIGS. 14A-M Show an example of cross-section views illustrating the fabrication process of the one-layer individual site on chip. FIG. 15N Shows an cross section view in the X direction of the one-layer individual site on chip. First, photolithography can be used to form the cavity pattern on the semiconductor wafer. Then the sensing materials are deposited by deposition. Last, etching of the sensing materials takes place.
An example embodiment of the process, which is not intended to limit the scope, is:
A). Single crystal Silicon (arranged with the “100” silicon surface) wafer ( [symbol] ) about several hundreds micrometers is used for the substrate of the array chip; Clean the silicon wafer for subsequent surface silicon oxidation;
B). Thermal wet oxidation is used for silicon oxide ( [symbol] ) (several micrometers) growth on the Si wafer. Silicon oxide is used for hand mask of silicon etching;
C). Several micrometers Photoresist ( [symbol] ) deposition on the Si wafer with several micrometers silicon oxide on the surface; Spin-coater is used to deposit the Photoresist.
D). Exposure and development to form the Photoresist stripe array with cavity using cavity mask; The Photoresist stripe array is used for mask of silica etching;
E). Silicon Oxide etch using patterned Photoresist stripe; Wet etching can be used in this process.
F). Remove Photoresist forming silicon oxide thin film with pattered silicon in silica cavity;
G). Silicon etch forming cavity with 45° inclined plane (Silicon (111) surface);
H). All silicon oxide thin film etch just left silicon wafer with cavity; Wet etching can be used in this process as the previous step.
I). Several hundreds micrometers Au ( [symbol] ) deposition to form Au or other metals electrodes;
J). Au thin film etch using electrode mask. Two methods (dry etch or wet etch) can be used to etch Au film
K). Gas sensitive nanomaterials ( [symbol] ) deposition using sensing materials mask;
L). Gas sensing materials etch using valley mask; Dry etch can be used to etch the sensing materials.
M). Cross-section views in the Y direction in FIG. 15M of one-layer individual site on chip after material etch.
N). Cross-sections of one layer individual site on chip after material etch.
Mask 1 is the cavity mask; Mask 2 is the electrode mask; Mask 3 is the material mask; Mask 4 is the valley sensing material pattern mask. The material mask can be the same as the cavity mask.
Au or other metals materials electrodes exposed deliberately for wire bonding to external current; voltage; capacitance; resonance frequency; resistance measurement.

### Multi-Layers:

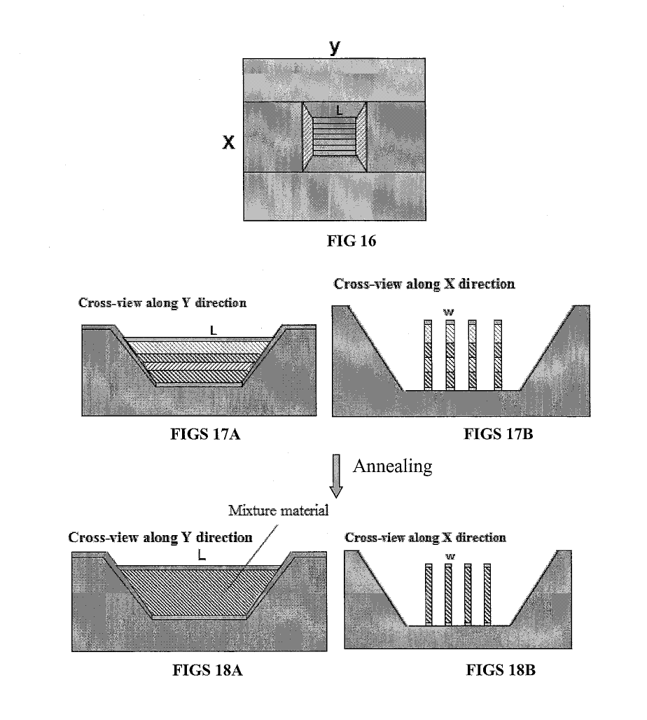

FIG. 16 Show top view of an example of the multi-layer of individual site on chip, X and Y are two directions that are perpendicular to one another in the horizontal plane; FIG. 17-18 Show schematic diagram illustrating the multi-layer sensing materials of the individual site on chip. The annealing process is used for getting a stable and mixture compound material. In the annealing process, the temperature is about 500° C., and the time can be about 2 hours, longer annealing time can be adopted. These four different colors stand for different sensing materials. There can be many more layers than four.

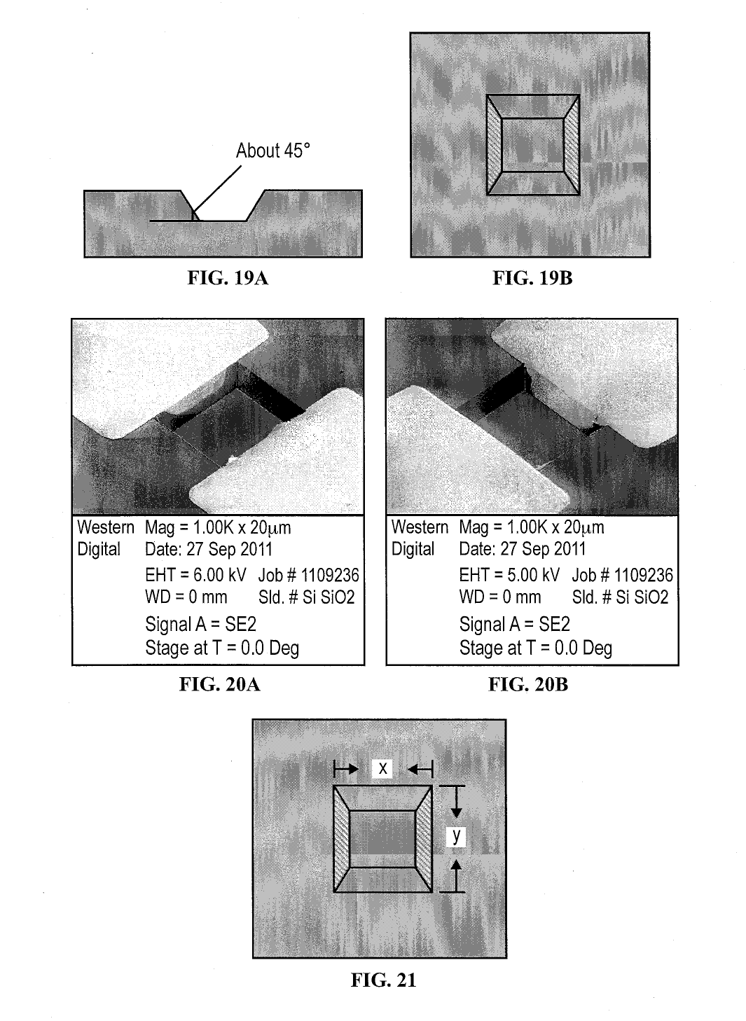

5.2.2 Cavity of Each Individual Site on Chip (Crystal Direction of Silicon is Exploited, Refer to Patent Reference or Reference)
Each individual site on chip has a cavity. The size, shape, depth and angle of cavity are all not limited, and can be changed.
(a). Angle:
   Angle is alterable
   Example:
FIG. 19A shows a cross-view of a cavity with 45° angle via etching the silicon with (100) crystal direction using patterned Photorisist.
FIG. 19B shows a top view of a cavity with 45° angle via etching the silicon with (100) crystal direction using patterned Photorisist.
   Wet etching using an alkaline etchant can be used to get this cavity with 45° angle. The resulting silicon surfaces exposed include the (100) silicon surface in the bottom horizontal surface and the (110) silicon surface about 45° angle at the side surface. Cavities with other angles (eg. 53.7° . . . ) and other silicon surface which can be get via changing the surface of silicon (111) substrate can also be used for our combinational array. So, angle is alterable and not limited to 45°. Different angles or shape can be obtained by choosing a different crystal direction of silicon or different etchants.
FIG. 20A-20B Scanning Electron Micrograph (SEM) images of cavity with 45° angle with gold electrode. White part is the gold electrode with ears; the gray part is the silicon substrate with cavity of 45° angle.
(b). Size:

The cavity (the length and the width) size is alterable.

FIG. 21 shows a top view of cavity with X length and Y width. The size is alterable and not limited. We can get different cavities with different sizes by changing the size of the cavity Mask and etching time.
1) Cavity mask dependent.
   The values of X and Y are either the same as each other or different to each other. It depends on the size of cavity mask.
   When X is the same as Y, we can change the size of the cavity by changing the values of “x” and “y”.
   When X is different to Y, we can change the size of the cavity by changing the value of “x” and “y” individually).
2) Etching time dependent.
   The size of cavity also can be changed via controlling the time of etching. Basically, the longer etching time is, the bigger the cavity size is.
   Example:
      Silicon etching: 33% KOH etch at 50 C

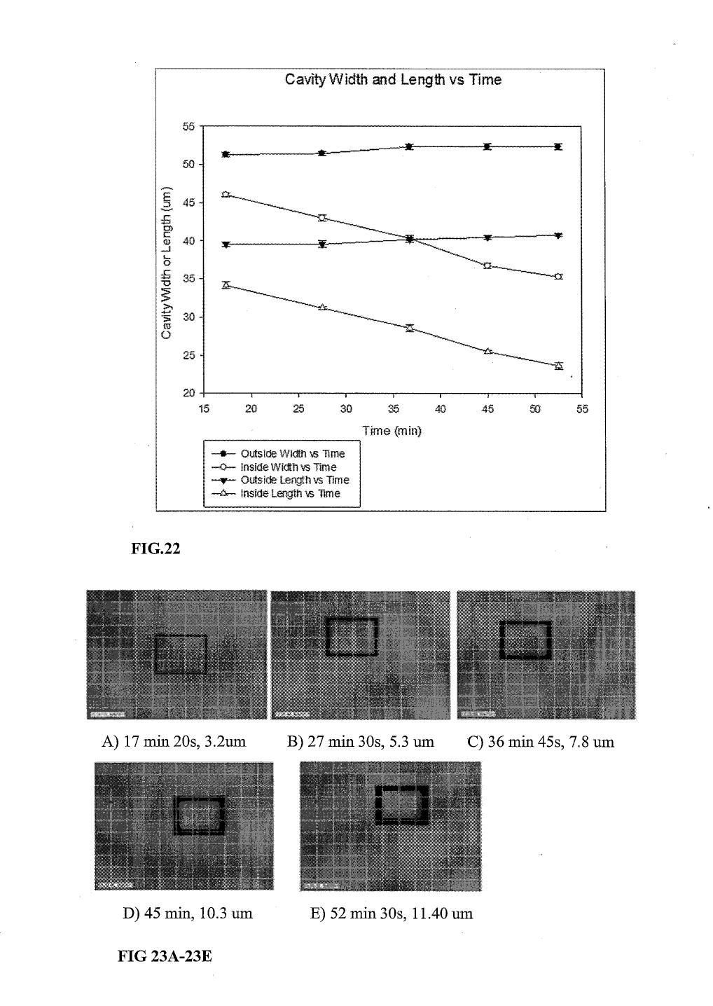

FIG. 22 shows an example of relationship schematic between silicon cavity width, length and etching time. Here, 33% KOH solution is used as the etching solution. From this picture, basically we can know that when we extend the etching time, the outside width and length of cavity both increase faintly, the inside width and length of cavity both decrease on the contrary. Other etching solution can be used as etching solutions. Different etching solution has different etching performances. So the size of cavity can be changed by controlling the etching time and changing the size of cavity mask.
(c). Depth:
Depth is alterable and not limited. We can get cavities with different depths via changing the time of silicon etching. Basically, the longer etching time is, the deeper the cavity is.

### Example:

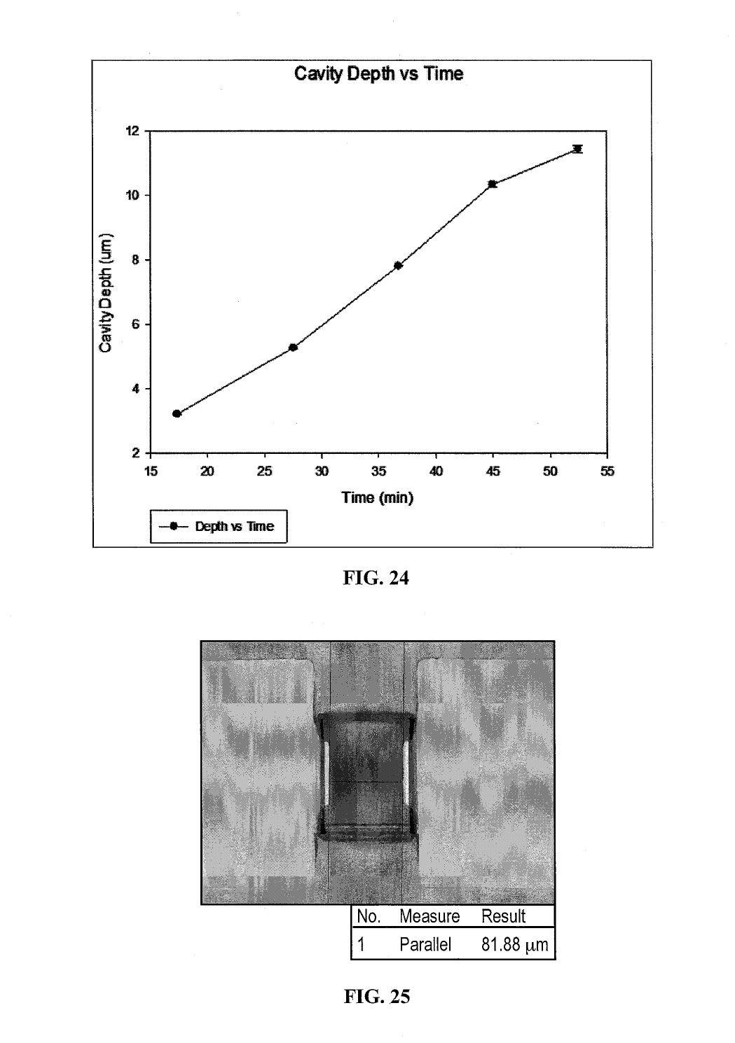

Silicon after KOH etching picture
33% KOH etch at 50° Silicon cavity depth pictures
FIG. 23A-23E Show different silicon cavities depth pictures etched by 33% KOH etching solution at 50° with different etching time. The violet part is the silicon substrate, and the black part is the angled interfaces. The larger area black part is, the deeper the cavity is. When the etching time is extended, the depth of cavity increase obviously. So we know that the longer etching time is, the deeper the cavity is. Other etching solutions can be used as etching solutions.
FIG. 24 shows an example of relationship schematic between silicon cavity depth and etching time. Here, 33% KOH solution is used as the etching solution. From this picture, basically we can know that when we extend the etching time, the depth of cavity increase obviously. Other etching solution can be used as etching solutions. Different etching solution has different etching performances. So the depth of the cavity can be changed via controlling the etching time as well.
(d). Shape:
The shape of cavity is not limited to square or rectangle, other basic shapes of cavity can also be used in the individual site, and it is determined by shape of the mask of cavity. A different shaped cavity is obtained by changing to a different shape cavity mask.

5.2.3 Electrode Ear-Type

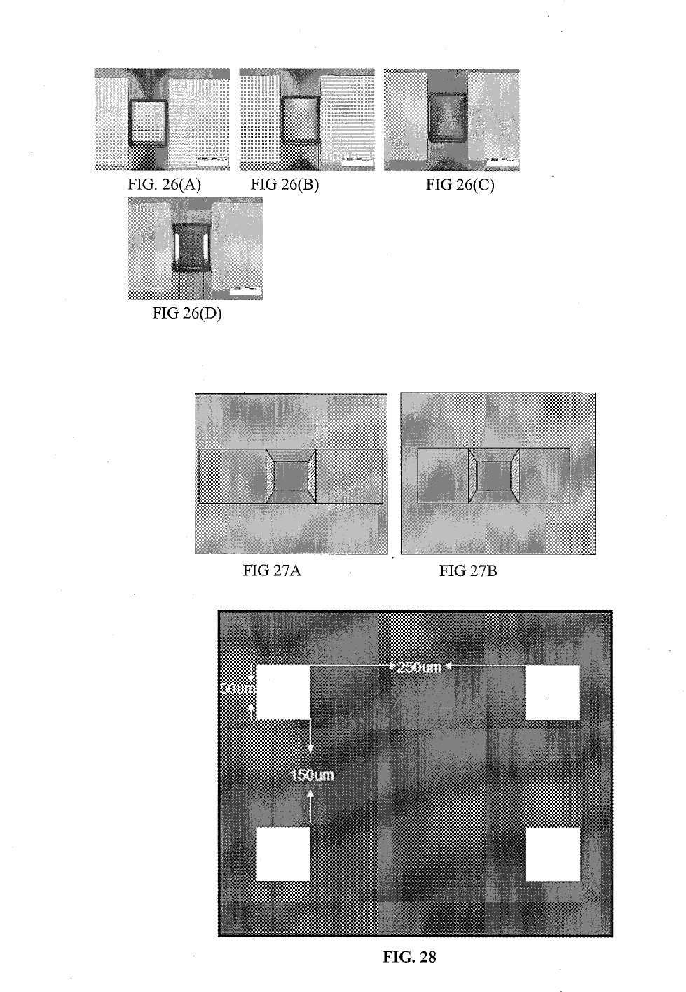

The shape of each electrode on individual site chip is ear-type shape. The size of electrode ears can be changed, and it is determined by the shape of the mask of electrode. So we can get different electrode with different shape by changing different shape cavity mask.
FIG. 25 Photon microscope picture of cavity with ear-type Au-electrode: The green part is the silicon substrate, and the black-green part is the cavity with angled interfaces. The golden part is the ear-type Au electrode. In this picture, there are two Au electrode ears in the cavity.
FIG. 26A-26D Show different width electrode ear pictures

| | | | |
|---|---|---|---|
| A) 0 um ear | B) 3 um ear | C) 5 um ear | D) 10 um ear |

Width of electrode ear is alterable. The width of electrode ear of electrode is determined by the shape of the mask of electrode. So we can get different electrode with different width of electrode ear by changing different shape cavity mask.

5.2.4 Square or Other Shapes for Electrode Bonding Side

FIG. 27A-27B Show different shapes of electrode bonding side, square and rectangle. The shape of electrode bonding side can not only be square, but be other shapes (rectangle . . . ). It can be changed by changing the electrode masks. The gray part is the silicon substrate; the golden part is the Au or other metal thin film used as electrodes.

5.2.5 Selection of Masks for Individual Site on Chip.

In an exemplary embodiment:
Cavity size is 50 um by 50 um;
Electrode size is 100 um by 100 um;
Ridge width ranges from several dozens nanometers to several micrometers.

### 5.2.5.1 Masks for Cavity

The pitch of four cavities is 200 um by 300 um. This mask will be used again for metal deposition and sensing materials deposition.
FIG. 28 Show an example of a cavity mask for lithography of positive photoresist, the white part of the mask is nonopaque part and the gray part is lightproof part. For lithography of positive photoresist, masks are the contrary. The pitch of four cavities can be changed. It can range from several dozen micrometers to several millimeters. It should match the electrode masks. Its minimum depends on the cavity mask size and the electrode mask size.

### 5.2.5.2 Masks for Electrodes

Electrode size is 100 um by 100 um
   The pitch of two electrodes is 200 um by 150 um
   The pitch of four electrodes is 300 um by 200 um

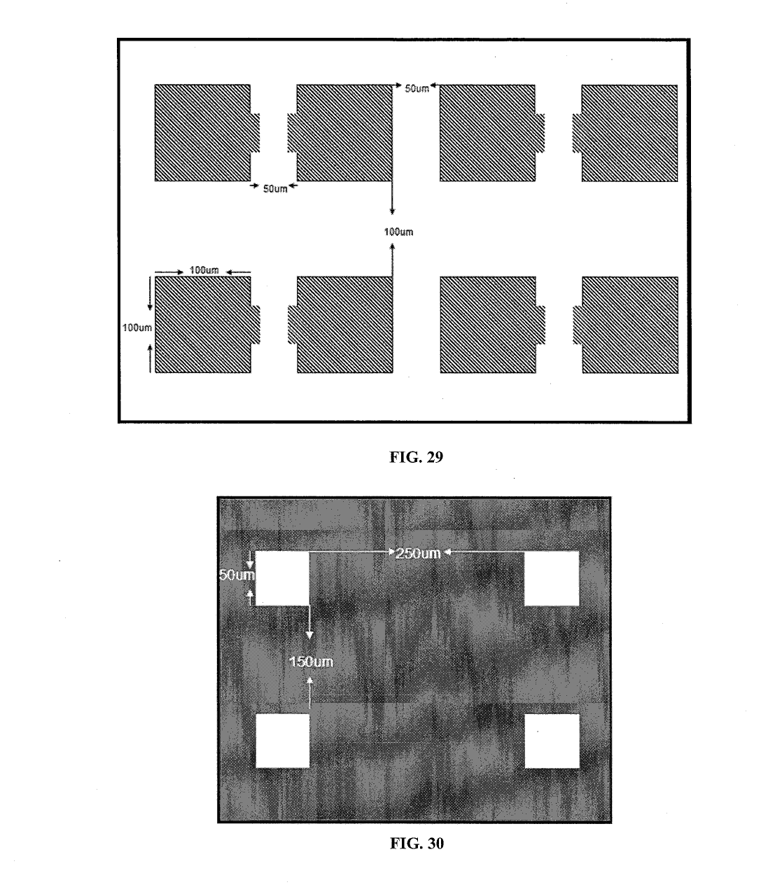

FIG. 29 Shows an example of electrode mask for lithography of positive photoresist. The white part of the mask is nonopaque part and the black shade part is lightproof part. For lithography of positive photoresist, masks are the opposite.

The pitch of four electrodes can be changed. It can range from several hundreds micrometers to several millimeters. It should mach the cavity masks. The pitch of two electrodes can be changed. Its minimum depends on the cavity mask size. The bigger the pitch of electrodes is, the greater the density of sensing units in each combinational array chip is.

### 5.2.5.3 Masks for Sensing Materials

Masks for sensing materials are the same as the Masks for the cavity

FIG. 30 Shows an example of Masks for sensing materials. This mask can be used for sensing materials deposition and cavity forming. It can be reused.

### 5.2.5.4 Masks for Ridge Pattern

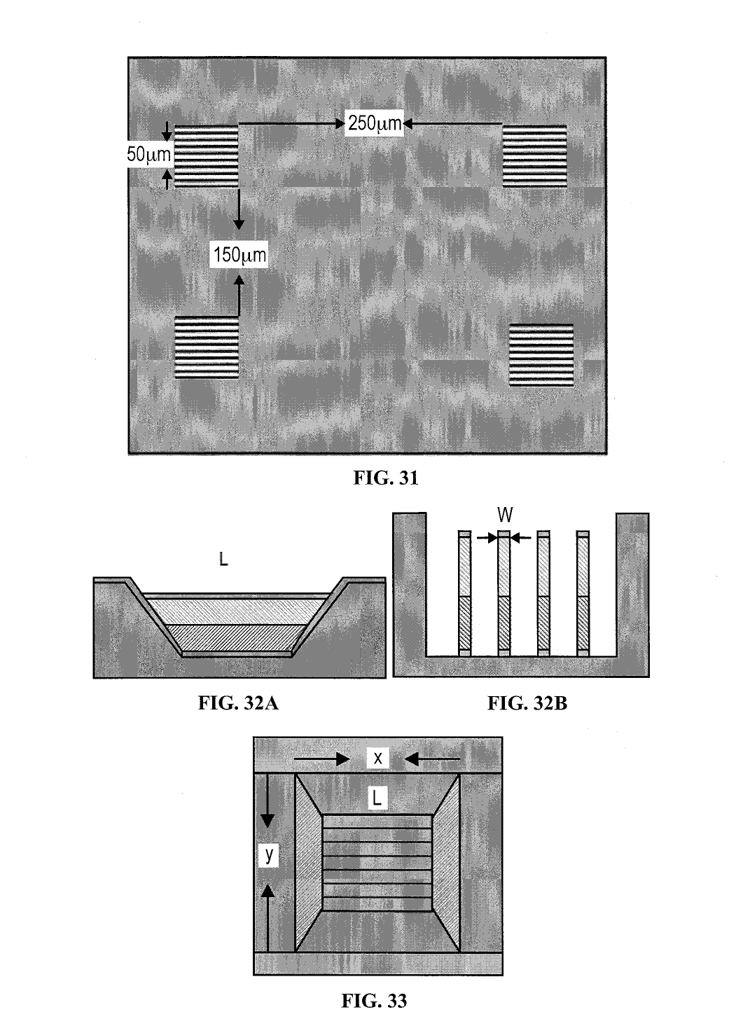

The pitch of four cavities is 200 um by 300 um. FIG. 31 Shows an example of small unit of Ridge Pattern mask for lithography of positive photoresist. The white part of the mask is nonopaque part and the black shade part is lightproof part. For lithography of positive photoresist, masks are the opposite. The width of each valley nanobelt is about several dozen nanometers to several micrometers. Repeat ridge & valley pattern to reach 50 um length.

5.2.6 Using ridge and valley method to create vertical nanobelt thin film with combinatorial power. The ridge shape of the nanobelt sensing materials array with varying thickness are designed and fabricated by combining silicon processing, MEMS technologies, photolithography and bulk micromachining techniques such as 33% KOH solution etching and RIE. The basic idea is to fabricate the nanobelt with a pattern array, replace the continuous sensing thin film in the sub-micron scale. This shape sensing materials have high performance of gas sensing. Two main processes in this patent are followed: a substrate patterning process and a ridge and valley sensing materials pattering process. These are simple process to get lots of different sensing materials with high performance.

### 5.3 Groove-Etching Method

Etch processes are judged by their rate, selectivity, uniformity, directionality (isotropic or anisotropic), etched surface quality, and reproducibility. The two most commonly employed etching methods use either liquid chemicals (wet etching) or reactive gas plasmas (dry etching). Wet etching has some advantages: simplicity, low cost, low damage to the wafer, high selectivity, and high throughput. But they have many limitations, including its isotropic nature, which makes it incapable of patterning sub-micron features, and the need for disposal of large amounts of corrosive and toxic materials. Dry-etching methods became the favored approach for the etching processes for integrated circuit manufacture. These use plasma-driven chemical reactions and/or energetic ion beams to remove materials. The advantage of dry over wet etching is that it provides higher resolution potential by overcoming the problem of isotropy. Other benefits are the reduced chemical hazard and waste treatment problems, and the ease of process automation and tool clustering.

Dry etching takes place through a combination of chemical and physical components in order to obtain the desired results. Some of the dry-etching techniques in common usage include:
Ion etching, Plasma Etching, Reactive-Ion Etching (RIB, DRIE), Reactive-Ion-Beam Etching (RIBE), Electron Cyclotron Resonance (ECR), Inductively Coupled Plasma (ICP). The basic methods we use are mainly RIE/DRIE. But other method are also can be used for etching materials. DRIE or RIE can be used to get the groove. The capabilities of RIE, mainly its independence on crystal orientation and the potential to fabricate arbitrarily shaped geometries, made plasma etching a promising candidate as a new microstructuring technique for the MEMS field. micromechanical elements containing shallower pattern features can be realized using conventional RIE approaches. We can use this dry etching (RIE or DRIE) to get valley nanobelt sensing materials for most of gas sensors.
   6. Interface of IC measurements. Various interconnections can be used. Those that provide less noise, better integration, better miniaturization, and faster signal processing are described herein.
   6.1 Through-Silicon Via (TSV) A through-silicon via (TSV) is a vertical electrical connection (via)(Vertical Interconnect Access) passing completely through a silicon wafer or die. TSVs are a high performance technique used to create 3D packages and 3D integrated circuits, compared to alternatives such as package-on-package, because the density of the vias is substantially higher, and because the length of the connections is shorter. TSV are preferred, though need not necessarily by used.
   6.2 Flip-chip interconnecting semiconductor devices, such as IC chips and Micro-electro Mechanical Systems (MEMS), to external circuitry with solder bumps that have been deposited onto the chip pads. The solder bumps are deposited on the chip pads on the top side of the wafer during the final wafer processing step. In order to mount the chip to external circuitry (e.g., a circuit board or another chip or wafer), it is flipped over so that its top side faces down, and aligned so that its pads align with matching pads on the external circuit, and then the solder is flowed to complete the interconnect. While not necessary, flip chip interconnecting is a preferred bonding approach.

### 7. Measurement Modalities:

There are many kinds of measurement modalities to get the sensitivity of gas sensor, because the interaction between the analyte in the surrounding gas phase and sensing materials is detected either as a change in electrical conductance, capacitance, or potential of the active element. The sensitivity of a particular sensor can be obtained by measuring the changes of Impedance, Resistance or Capacitance.

### 7.1 Impedance

One principle of the sensor operation is the oxidation or the reductive reaction caused by gas molecules with the film surface. The electrical resistance of the sensor changes by this reaction. It is possible to operate as a sensor of the impedance change type by measure the impedance change of the electric characteristics of the sensing device. The sensor functions as impedance changeable sensor by a conductivity change and a permittivity change of the sensing film. Those changes are caused by the physical and chemical adsorption of gas molecules. So we can get the sensitivity of the sensor device and the response characteristic of the sensor by measure the impedance change.
Impedance is represented as a complex quantity Z. It is well known that the electrical behavior of the sensing materials can be analyzed using impedance plots, in which the impedance is shown in a complex plane with the reactance, imaginary part of impedance, plotted against the resistance, real part of impedance. Impedance is represented as a complex quantity Z.
It is well known that the electrical behavior of the sensing materials can be analyzed using impedance plots, in which the impedance is shown in a complex plane with the reactance, imaginary part of impedance, plotted against the resistance, real part of impedance. Impedance is represented as a complex quantity Z:

Resistive: Z_{R}=R

Inductive: Z_{L}=sL where s is the complex Laplacian frequency

Capacitive: Z_{c}=1/sC
For DC, s=0.
For AC, or steady state sinusoidal excitation, s=jω where ω=2πf.
Today's sensors typically only measure DC resistance, or capacitive reactance, as a quasi-specific function of analyte concentration. In addition to resistance and capacitance, the present invention also measures the spectra of complex impedance over several variables including temperature and electric field strength. The resultant data represent a multi-dimensional profile, or fingerprint, of the analyte(s) with much greater specificity. This multidimensional spectroscopic impedance analysis examines more of the physical parameters of the analyte(s) than possible with simple resistance or capacitance. The complex impedance spectra depend on a number of parameters including:
molecular weight and polarity (of the charged moiety if any) and resonant frequency
resonance vs electric field strength
impedance vs electric field strength
dielectric properties

So we can get the sensitivity of the sensor device and the response characteristic of the sensor by measuring the impedance change and get the change of the resistance.

### 7.2 Resistance

One principle of the sensor operation is the oxidation or the reductive reaction caused by gas molecules with the film surface. The resistance of the sensor changes by this reaction. So we can get the sensitivity of the sensor device and the response characteristic of the sensor by measure the change of resistance.

R=ρ*L/A
ρ - - - Electrical resistivity
L - - - The length of material
A - - - The cross sectional area

### Example:

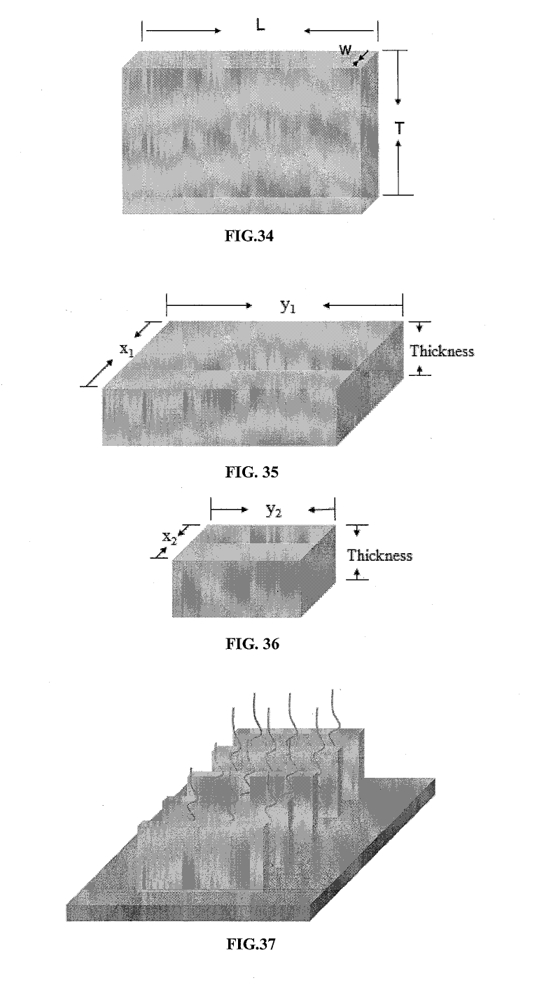

FIG. 32A-32B Cross-section of two layer individual unit of combinational array sensor. FIG. 33 Top view of two layer individual unit of combinational array sensor. FIG. 34 shows a 3D view of one valley of sensing material in cavity of individual unit on chip.

### Influence Factors:

R_{T}=R_{b}Rₛ*Rₑ
Where R_{T} is the total resistance of the sensor device; R_{b} is the bulk resistance of the sensor; Rₛ is the wire bonding resistance; Rₑ is the effect resistance.

R_{b}ρ*L/A

A=x*T

R=ρ*y/x*T

That L is the length of sensing material in cavity; S is the area of contact between sensing material and electrode; T is the thickness of sensing material in cavity;
a). Thickness (T)
   Dependence of thickness. Independence of the size of X, Y dimensions (width and length) or the size of cavity.
   FIG. 35 Shows one example of whole sensing material in cavity with the width is x₁ and the length is y₁
   FIG. 56 Shows one example of whole sensing material in cavity with the width is x₂ and the length is y₂

R_{T}=R

R=ρ*L/A

A=x*T

R=ρ*y/x*T
So R=ρ*A′*T (A′ is an definite value)

| | |
|---|---|
| Size of Cavity (x/y) | Independent |
| Length (y) | Independent |
| Width (y) | Independent |
| Thickness (T) | Dependence |

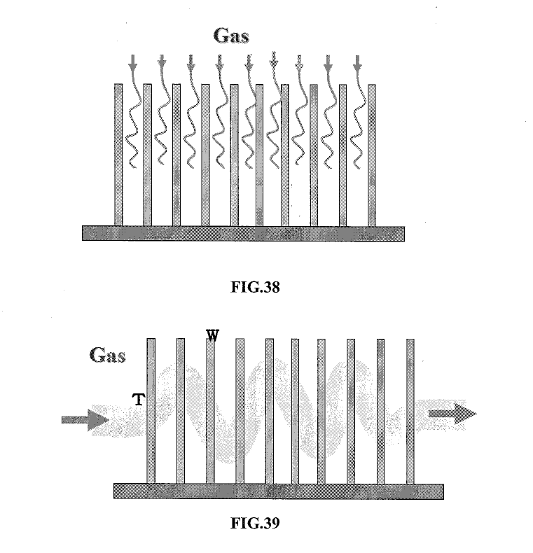

b). the diffusion of gases
   The principle of the change of electrical resistance is the interaction between the analytes. So the change of the resistance depends on the behavior of gas.
   FIG. 37 Show the 3D view of the diffusion of gas between vertical sensing materials. FIG. 38 Show the side view of the diffusion of gas between vertical sensing materials. FIG. 39 Show the side view of the diffusion of gas between vertical sensing materials.
   The mass of the diffused gas in limited time can be described by the following:

$$dM = - {D ( \frac{d ρ}{d x} )}_{{x}_{0}} Δ Sdt$$

Where the M is the mass of the diffused gas; D is the diffusion coefficient (which is a fixed value);

$${( \frac{d ρ}{d x} )}_{{x}_{0}} .$$

is the Gas density gradient; ΔS
is the contact area between gas and sensing materials; t is the time of diffusion.

$${( \frac{d ρ}{d x} )}_{{x}_{0}} \cdot ∝ [ gas ]$$

(The concentration of gas)
ΔS is the contact area between gas and sensing materials, it is parameters
depending on the surface volume ratio.

ΔS∝T/W

S=ΔR/R=A·[gas]^{B}
Where A and B are parameters depending on the working temperature, the contact surface (the surface volume ratio) and the gas adsorption mechanism (the diffusions of gas).
   So:

| | |
|---|---|
| [gas] | Dependent |
| T | Dependent |
| W | Dependent |

### 7.3 Capacitance

For detection of various chemical species, several transduction principles associated with the sensing approaches showing promise are based on metal oxides, acoustic waves, cantilever resonance, resistance or capacitive changes. The last class of sensors, the capacitive ones, is dominated by a) devices where the variations in device capacitance result from the change of dielectric permittivity of a chemically sensitive material.
   We can get the sensitivity of one sensor by measure the changes of Capacitance. Capacitive gas sensor is dominated by devices where the variations in device capacitance result from the change of dielectric permittivity of a chemically sensitive material.

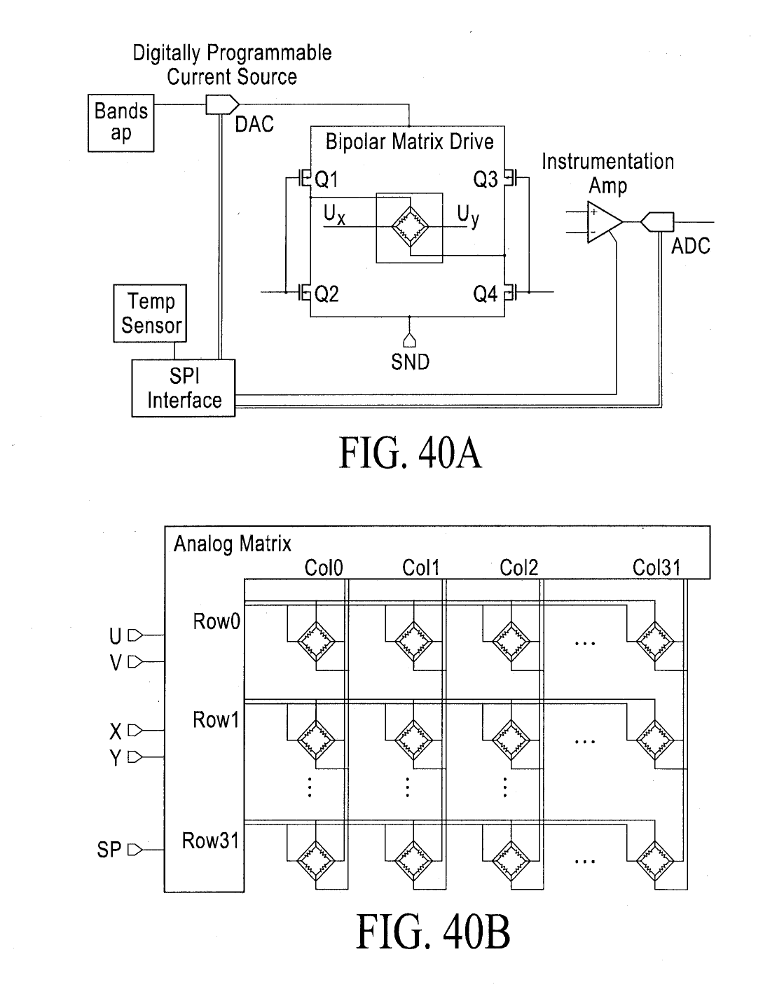

C=∈*A/d
The effect of frequency of measurement, v, on the response of the sensor array was tested upon exposure to various vapor concentrations, cg (ppm), of water and ethyl acetate. The difference in the dielectric constants of the two analytes (8=80 and 6, respectively), in conjunction with the different sorptive capacities of the various polymeric materials used, enabled us to test the array's performance in a range of ΔC responses covering ˜three orders, of magnitude.
Sensor Matrix.
   FIGS. 40A-B illustrate a sensor matrix and output circuit relating thereto. FIG. 40A illustrates an H-bridge sensor cell of 4 connected sensors and circuit elements as shown connected thereto, whereas FIG. 40B illustrates a sensor matrix of the macro cells. Not shown in the block diagram is a PID controlled heater, though in certain embodiments the heater is not needed. The PID heater element tightly coupled to the sensor array and typically be a platinum wire, or similar (nickel chromium, nickel nitride/aluminum nitride, etc.) heating element. The element raises the sensor temp, dwells for some time, then is allowed to cool to the next temp step. The sensor array and heater are electrically connected to an interface chip, but the two chips are thermally isolated. The interface chip provides for the detection and transmission of the output signals.

As shown in FIG. 40B, four sensor cells are arranged into a Wheatstone bridge. Two of the four sensors are masked (not exposed to the gas analyte) forming fixed resistors and balancing the bridge. An M×N array is formed with two row and two col analog MUXs (32×32 shown). Referring to FIG. 40A, the array (shown as a single macrocell) is set into an H-bridge and current-fed by a digitally controlled current source. The H-bridge allows for bipolar drive to cancel amplifier offsets. Finally, the bridge output is connected to a digitally-programmable instrumentation amp and to an ADC. The reading algorithm is:

1. select a macro cell (select row, col)
2. select H-bridge polarity 0
3. start at lowest instrumentation amp gain, take a reading. If necessary, increase gain,
repeat reading.
4. select H-bridge polarity 1
5. repeat reading, (calculate true bridge reading, remove DC offset)

The combinatorial array described herein is better for gas sensing for whole host or reasons, including the following combination of parameters that

### 1. Thickness

### 2. Porosity

### 3. Composition

### 4. Doping

### 5. Layers

6. Low work temperature

### 7. Resistance

Combination parameters (thickness, porosity, composition, layer, Low work temperature, Resistance)
There are many advantages about combination array sensors compared to single sensor. Reasons that why combinatorial array is better for gas sensing include:

### a) Easily Changeable Thickness of Sensing Materials

Sensitivity of this kind of sensor device is dependent on thickness of material, so we can change the thickness of material to get the best performance of device easily.

### b) High Porosity.

Large surface volume ratio can increase the chance of contaction between gas and the surface and enhance the interaction of them. This kind of valley array sensor is familiar to the nanostructure sensor. It is a good material for gas sensor because its porosity enhances their surface volume ratio and also it can reduce the work temperature of sensor.

### c) Composition

Compound materials have higher sensitivity than the single material has. There are many different kinds of Compound materials we can get for this. There are many parameters which can be changed to get lots of kinds of compound materials (Number of layers, the width of thin film nanosheet, the thickness of sensing materials, kinds of materials, compound modes of the same materials, the size of combinational array and so on.

### d) Doping

The catalytic activity of MOX nanoparticles can be improved by metal ion dopants. Doping can be used to influence the band gap energy etc.

### e) Layers

There are many layers in one individual unit of sensor, we can increase the number of layer to get more integrated materials with each sensing material have its special response to one or more gas.

### f) Low Work Temperature.

On the other hand use of nanomaterials for the sensing device for their enormously increased surface to volume ratio compared to their bulk counterpart leads to opportunities to lower the operating temperature of metal oxide semiconductor gas sensors.
This compound mode of combination array sensor can detect different kind of gases with perfect performances simultaneously and we can get complicated Combinational Array using simple method
   Owing to the fact that there are many advantages about combination array sensors compared to single sensor, such sensors can be manufactured as portable devices that can be operated at elevated temperature by battery power and used in a large variety of applications, such as fire detectors, leakage detectors, controllers of ventilation in cars and airplanes, and alarm devices warning that concentrations of hazardous gases have exceeded preset thresholds in workplaces. They can even be used for the detection of smells generated from food or household products and for analysis of complex environmental mixtures.

Although the present invention has been particularly described with reference to embodiments thereof, it should be readily apparent to those of ordinary skill in the art that various changes, modifications and substitutes are intended within the form and details thereof, without departing from the spirit and scope of the invention. Accordingly, it will be appreciated that in numerous instances some features of the invention will be employed without a corresponding use of other features. Further, those skilled in the art will understand that variations can be made in the number and arrangement of components illustrated in the above figures.

1. An apparatus for measuring a concentration of at least one gas in air comprising:
an integrated semiconductor sensor unit, the semiconductor sensor unit comprising:
a common substrate;
a plurality of semiconductor sensors disposed over the common substrate,
wherein each of the plurality of semiconductor sensors senses at least one of a plurality of different gases, wherein at least one of the plurality of sensors senses the at least one gas, and wherein each of the plurality of the semiconductor sensors include two electrodes and a plurality of semiconductor ridges disposed between the two electrodes, each of the plurality of semiconductor ridges being made of a same composition of semiconductor material, thereby allowing the air with the gas disposed therein to be proximate to each of the plurality of semiconductor ridges unless inhibited by an inhibitor material; and
a circuit that uses a source current to pass a measurement current through at least some of the plurality of semiconductor sensors and cause outputting of at least one measurement signal from the plurality of semiconductor sensors.

2. The apparatus according to claim 1 wherein different ones of the plurality of semiconductor sensors have different semiconductor materials.

3. The apparatus according to claim 2 wherein the plurality of semiconductor sensors each has one layer of semiconductor material.

4. The apparatus according to claim 3 wherein some of the plurality of semiconductor sensors has a first semiconductor material and others of the plurality of semiconductor sensors has a second semiconductor material different from the first semiconductor material.

5. The apparatus according to claim 2 wherein the plurality of semiconductor sensors each has at least two layers of semiconductor material and wherein the plurality of semiconductor sensors are arranged in an array.

6. The apparatus according to claim 5 wherein
for a first layer, some of the plurality of semiconductor sensors in a first row have a first semiconductor material and others of the plurality of semiconductor sensors in a second row have a second semiconductor material different from the first semiconductor material, and
for a second layer, some of the plurality of semiconductor sensors in a first column have a third semiconductor material and others of the plurality of semiconductor sensors in a second column have a fourth semiconductor material different from the third semiconductor material,
such that there exist at least four different semiconductor sensors that can sense different gases.

7. The apparatus according to claim 6 wherein the circuit includes an address circuit that addresses different ones of the plurality of semiconductor sensors at different times.

8. The apparatus according to claim 1 wherein:
the plurality of semiconductor sensors includes at least two semiconductor sensors that are connected together in a bridge, such that the two semiconductor sensors are comprised of the same semiconductor material and sense the same gas, wherein a first of the semiconductor sensors is exposed to air with the gas disposed therein, and wherein a second of the semiconductor sensors is not exposed to air with the gas disposed therein using the inhibitor material; and
the circuit outputs two different measurement signals, a first measurement signal taken the first semiconductor sensor based upon one polarity of the source current and a second measurement signal taken from the second semiconductor sensor based upon an opposite polarity to the one polarity of the source current.

9. The apparatus according to claim 1 wherein:
the plurality of semiconductor sensors includes at least four semiconductor sensors that are connected together in a bridge, such that the four semiconductor sensors are comprised of the same semiconductor material and sense the same gas, wherein a first and third opposite two of the semiconductor sensors are exposed to air with the gas disposed therein, and wherein second and fourth other opposite two of the semiconductor sensors are not exposed to air with the gas disposed therein using the inhibitor material; and
the circuit outputs two different measurement signals, a first measurement signal taken the first semiconductor sensor based upon one polarity of the source current and a second measurement signal taken from the second semiconductor sensor based upon an opposite polarity to the one polarity of the source current.

10. A method of making a semiconductor gas sensor comprising the steps of:
providing a substrate:
opening a cavity in the substrate;
filling opposite sidewalls of the cavity and an adjacent top region with a conductor to form a pair of electrodes; and
forming a plurality of semiconductor ridges disposed between the two electrodes within the cavity, each of the plurality of semiconductor ridges being made of a same composition of semiconductor material, thereby allowing the air with the gas disposed therein to be proximate to each of the plurality of semiconductor ridges.

11. The method according to claim 10 wherein the method of forming the semiconductor gas sensor forms a plurality of semiconductor has sensors, such that:
the step of opening the cavity opens a plurality of cavities;
the step of filling the opposite sidewalls fills the opposite sidewalls and the adjacent top region of each of the cavities to form a pair of electrodes for each cavity;
the step of forming the plurality of semiconductor ridges occurs within each cavity.

12. The method according to claim 11 wherein different ones of the plurality of semiconductor sensors have a different composition of semiconductor materials.

13. The method according to claim 12 wherein the plurality of semiconductor sensors each has one layer of semiconductor material, and wherein, during the step of forming the semiconductor ridges, there is included the steps of:
forming some of the plurality of semiconductor sensors with a first semiconductor material; and
forming others of the plurality of semiconductor sensors with a second semiconductor material different from the first semiconductor material.

14. The method according to claim 12 wherein the plurality of semiconductor sensors each has at least two layers of semiconductor material and wherein the plurality of semiconductor sensors are arranged in an array, and wherein, during the step of forming the semiconductor ridges, there is included the steps of
forming, in a first layer, some of the plurality of semiconductor sensors in a first row with a first semiconductor material and others of the plurality of semiconductor sensors in a second row with a second semiconductor material different from the first semiconductor material, and
forming, in a second layer disposed over the first layer, some of the plurality of semiconductor sensors in a first column with a third semiconductor material and others of the plurality of semiconductor sensors in a second column with a fourth semiconductor material different from the third semiconductor material,
such that there exist at least four different semiconductor sensors that can sense different gases.

15. A method of forming a semiconductor ridge having a predetermined composition and a predetermined length, width and depth for use as a gas sensor comprising the steps of, comprising the steps of:
forming a first layer of semiconductor material of a predetermined material to a predetermined thickness on a substrate;
forming a second layer of semiconductor material of another predetermined material that is different than the first predetermined material to another predetermined thickness over the first layer of semiconductor material to form a composite layer;
etching the composite layer to form the semiconductor ridge having the predetermined length, width, and exceeding the depth desired for the semiconductor ridge; and
removing the semiconductor ridge from the substrate so that the semiconductor ridge results in the predetermined depth.

16. A method of measuring a concentration of at least one gas in air comprising:
introducing air into a semiconductor sensor unit;
disposing the air proximate to a plurality of sensors within the semiconductor sensor unit, each of the sensors including a plurality of semiconductor ridges, the plurality of semiconductor ridges for each sensor being formed over a common substrate, parallel to each other and having opposite ends, with each connected between a pair of electrodes at the opposite ends thereof, each of the plurality of semiconductor ridges being made of a same composition of semiconductor material;
obtaining a plurality of measurement signals from the plurality of semiconductor sensors using a circuit that passes a measurement current through the plurality of semiconductor sensors and cause outputting of the plurality of measurement signals; and
analyzing the measurement signals using a detection algorithm to determine a concentration of the gas.
